(19) United States
(12) Patent Application Publication
(10) Pub. No.: US 2023/0404650 A1
(43) Pub. Date: Dec. 21, 2023

(54) SCISSOR STYLE VESSEL SEALER WITH SQUEEZE ACTIVATED TRANSECTION

(71) Applicant: Conmed Corporation, Largo, FL (US)

(72) Inventor: Aaron SZABO, Swanton, OH (US)

(73) Assignee: Conmed Corporation, Largo, FL (US)

(21) Appl. No.: 18/251,073

(22) PCT Filed: Nov. 3, 2021
(86) PCT No.: PCT/US2021/057872

§ 371 Date: Apr. 28, 2023

Related U.S. Application Data
(60) Provisional application No. 63/108,965, filed on Nov. 3, 2020.

(51) Int. Cl. A61B 18/14

(52) U.S. Cl. CPC A61B 18/1442; A61B 2018/00607

(57) ABSTRACT

A scissor-style vessel sealing having a knife engagement mechanism that will automatically drive a knife between the jaws when the handles are moved into the closed position. The vessel sealer has a first shaft with a first jaw and a second shaft having a second jaw pivotally coupled to the first shaft so that the jaws are moveable between open and closed positions. A knife actuating assembly is coupled to the first shaft to move between a first position, where the knife is in a retracted position, to a second position, where the knife is in an extended position, in response to contact by the second shaft when the scissor-style vessel sealer is closed. A knife lockout may be used to disengage the knife engagement mechanism from the knife to prevent movement of the knife when the scissor-style vessel sealer is closed.

### BACKGROUND OF THE INVENTION

### 1. Field of the Invention

The present invention relates to surgical instruments and, more specifically, to a scissor-type vessel sealer having a knife that can be driven by the scissoring action of the handles.

### 2. Description of the Related Art

Electrosurgical vessel sealers are used for the occlusion of blood vessels and halting of bleeding during surgical procedures. The jaws of the vessel sealer are interconnected to an electrosurgical generator that can selective supply radiofrequency (RF) energy to the jaws for the desiccation and sealing of a blood vessel that has been clamped between the jaws. Conventional vessel sealers have jaws that can hinge in response to user activation. For example, the jaws may be positioned on the ends of shafts that are connected to a common hinge pin so that the jaws scissor in response to user movement of handles or finger grips positioned at the other ends of the shaft. Most vessel sealing devices can also divide tissue with a knife that the surgeon can deploy manually by manipulating a trigger. Upon deployment with the jaws in a clamped position, the knife travels down the center of both jaws through a knife track. These devices can be cumbersome to use, however, as they require that the user operate the handles of the device to close the jaws and then operate a separate trigger to accomplish cutting with the knife while keeping the scissors closed. According, there is a need in the art for a scissor-style vessel sealer that can more easily allow for cutting of tissue trapped in the jaws of the sealer.

### BRIEF SUMMARY OF THE INVENTION

The present invention is a scissor-style vessel sealer than can automatically operate a knife when the vessel sealer is closed around tissue to be severed. The device includes a first shaft having a first jaw positioned at a first end and a second shaft pivotally coupled to the first shaft and having a second jaw associated with a second end so that the first shaft and second shaft are pivotal between an open position, where the jaws are spaced apart, and a closed position, where the jaws are closed. A knife actuating assembly is coupled to the first shaft and positioned to move between a first position, where the knife is in a retracted position, to a second position, where the knife is in an extended position, in response to contact by the second shaft when the first shaft and the second shaft are pivoted from the open position to the closed position. The knife actuating assembly may comprise a linkage extending between the first shaft and a knife pin secured to the linkage. The knife pin may be positioned in a slot extending longitudinally along the first shaft. The linkage may include a first leg pivotally coupled to the shaft by a first pivot and a second leg coupled to the first leg by a second pivot. The second leg of the linkage may be coupled to the knife pin.

In another embodiment, the second shaft of the scissor-style vessel sealer includes a first section pivotally coupled to a second section. A spring interconnects the first section and the second section to prevent pivotal movement of the first section relative to the second section when the first jaw and the second jaw are in the open position. When the first jaw and the second jaw are in the closed position, the first section and the second section are allowed to pivot relative to each other so that the second handle can engage the knife actuating mechanism.

In a further embodiment, the scissor-style vessel sealer includes a knife lockout interconnected to the knife actuating assembly that is moveable to selectively prevent or allow the knife actuating assembly to move the knife between the retracted position and the extended position. The knife lockout includes an interlock secured to a knife lockout button that is accessible by a user. The knife lockout is pivotally mounted to the first shaft for movement between a first position where the interlock is in engagement with the knife actuating assembly and a second position where the interlock is out of engagement with the knife actuating assembly. The interlock includes a detect and the knife actuating assembly includes a notch into which the detent may fit. The detent is positioned in the notch when the first jaw and the second jaw are in the open position to prevent movement of knife.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a side view of a scissor-style vessel sealer having a knife engagement mechanism according to the present invention in an open position.

FIG. 2 is a side view of a scissor-style vessel sealer having a knife engagement mechanism according to the present invention in a partially closed position.

FIG. 3 is a side view of a scissor-style vessel sealer having a knife engagement mechanism according to the present invention in a closed position.

FIG. 4 is a partial cutaway side view of a scissor-style vessel sealer showing the jaws in the closed position.

FIG. 5 is a side view of a scissor-style vessel sealer showing an approach for allowing the handles to be further closed after jaws are in the closed position along with a callout showing an internal breakaway spring according to the present invention.

FIG. 6 is a partial cutaway side view of a scissor-style vessel sealer showing the jaws in the closed position and the knife engagement assembly is the retracted position.

FIG. 7 is a partial cutaway side view of a scissor-style vessel sealer showing the jaws in the closed position and the knife engagement assembly in the extended position.

### DETAILED DESCRIPTION OF THE INVENTION

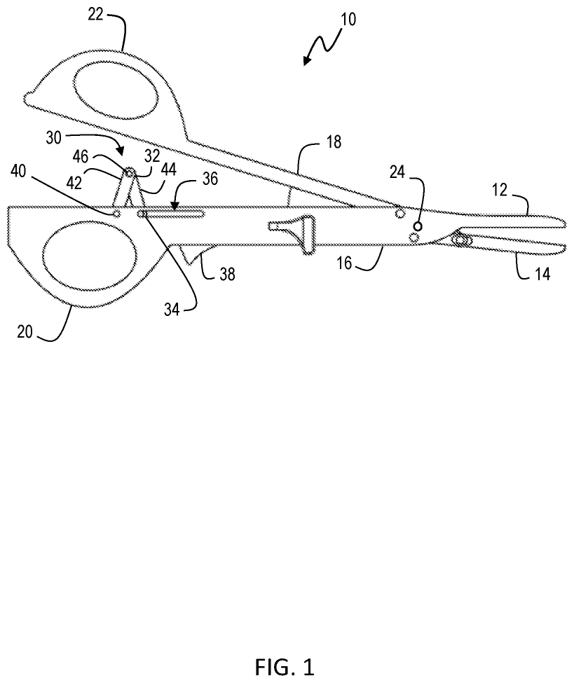

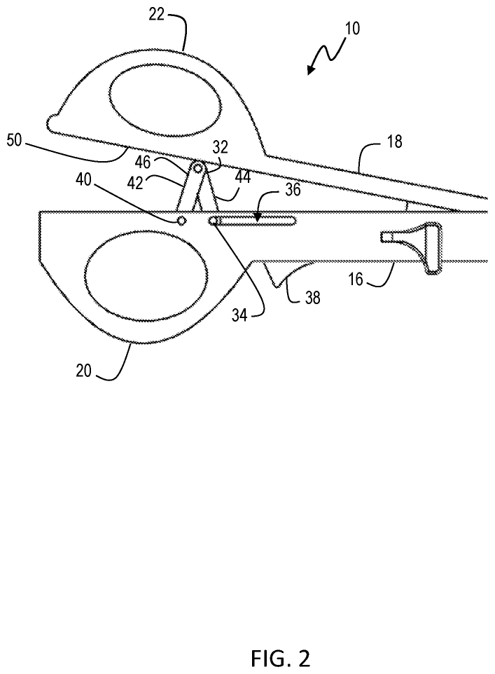

Referring to the figures, wherein like numeral refer to like parts throughout, there is seen in FIGS. 1 and 2 a vessel sealer 10 according to the present invention. Vessel sealer 10 comprises a pair of jaws 12 and 14, each of which is mounted to an end of a corresponding shaft 16 and 18. The opposing ends of shafts 16 and 18 include user handles 20 and 24 defining finger holes therethrough to allow for easy grasping by a user. Shafts 16 and 18 are pivotally coupled to each other at an intermediate portion thereof by a pivot 24. As should be recognized by those of skill in the art, vessel sealer 10 is operated by driving the user handles 20 and 22 in a scissoring motion to selectively open and close jaws 12 and 14. As is known in the art, an electrosurgical controller may be mounted to shaft 18 to provide radiofrequency (RF) energy to jaws 12 and 14 and to allow a user to selectively energize jaws 12 and 14 to perform electrosurgical operations when jaws 12 and 14 are closed about tissue to be treated. As seen in FIG. 1, vessel sealer 10 further includes a knife engagement assembly 30 comprising a linkage 32 coupled to a knife pin 34 positioned in a track 36 formed in shaft 16. A knife lockout button 38 is coupled to linkage 32. Linkage 32 is coupled to shaft 16 by a first linkage pivot 40, and includes a first leg 42 extending from first linkage pivot 40 and coupled to a second leg 44 by a second linkage pivot 46. Second leg 44 is coupled to knife pin 34.

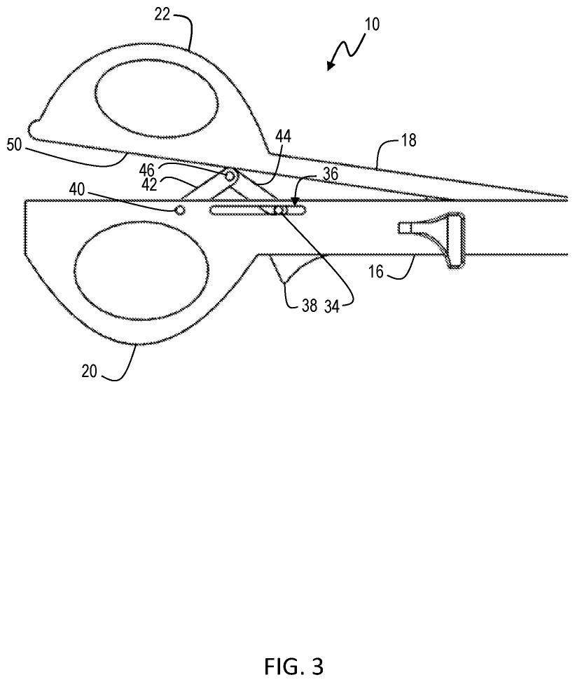

Referring to FIG. 2, partial closing of vessel sealer 10 by scissoring of handles 20 and 22 will result in the inner surface 50 of handle 22 applying a force to linkage 32. Referring to FIG. 3, further closing of vessel sealer 10 causes linkage 32 to extend via pivoting of first leg 42 and second leg 44 so that linkage 32 moves from a retracted position to an extended position, thereby driving knife pin 34 longitudinally along track 36 toward jaws 12 and 14. Linkage 32 is further coupled to a knife driver 52 that extends within shaft 16 and forms knife 54. As a result, scissoring of vessel sealer 10 into the jaws closed position will advance knife 54 between jaws 12 and 14 to sever any tissue trapped therebetween.

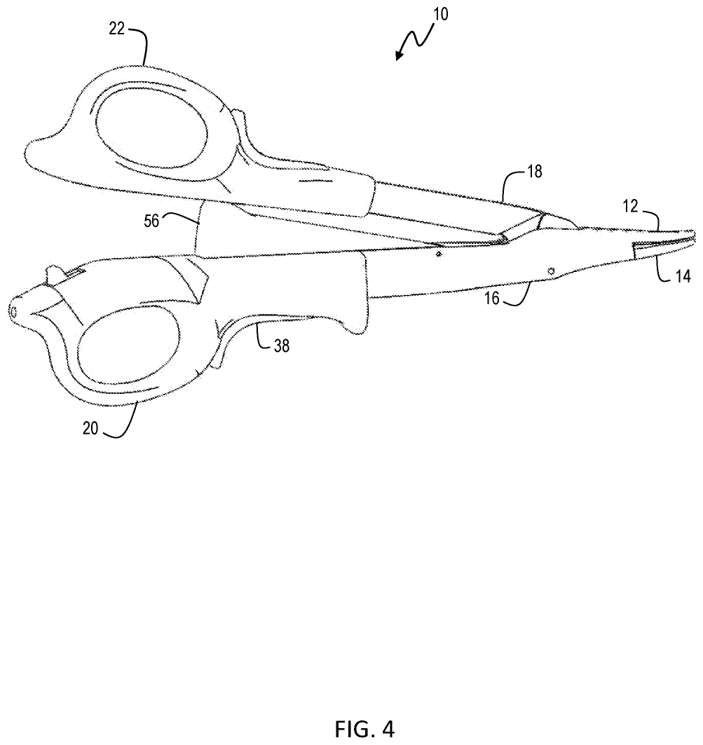

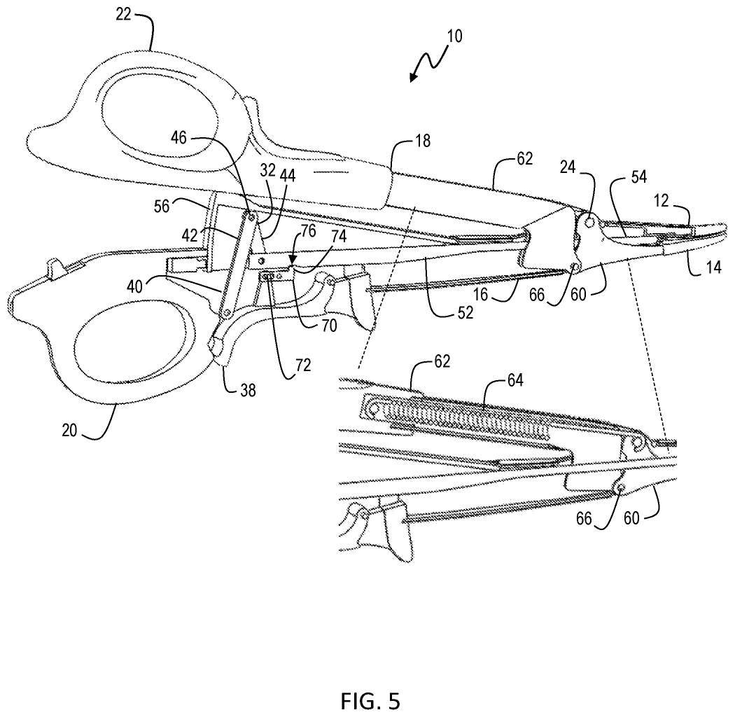

Referring to FIG. 4, a cover 56 encloses linkage 32 and provides a surface that handle 22 may contact to apply the force to linkage 32. As seen in FIG. 5, cover 56 bears directly on linkage 32 so that linkage 32 will move between the retracted and extended positions in response to scissoring of handles 20 and 22. As seen in FIG. 5, contact between handle 22 and cover 56 occurs once jaws are at least partially closed and, preferable, completely closed. Further scissoring of handles 20 and 22 to drive knife 54 is accomplished by having shaft 18 split into a first section 60 coupled to jaw 14 and a second section 62 coupled to handle 22. First section 60 and second section 62 are connected to each other via a second pivot 66 and a breakaway spring 64 that biases first section 60 and second section 62 into a longitudinally aligned position such as that seen in FIG. 5. Further scissoring of handles 20 and 22 thus allows handle 22 to move toward handle 20 as first section 60 and second section 62 pivot about second pivot 66 against the bias of breakaway spring 64 about a second pivot 66 when jaws 12 and 14 are closed so that handle 22 can engage cover 56 to drive linkage 32 into the extending position so that knife 54 advanced between jaws 12 and 14 while they are in the closed position. The biasing force of breakaway spring 64 may be selected to release first section 60 from second section 62 and thus drive knife 54 before the force being applied to jaws 12 and 14 by scissoring of handles 20 and 22 will damage vessel sealer 10. As an alternative to second pivot 66, first section 60 and second section 62 may be coupled together by a flexible link that will not flex until handle 22 contacts cover 56.

Referring to FIG. 5, knife lockout button 38 may be coupled to an interlock 70 that is biased to selectively engage and thus lock knife driver 52 against movement unless knife lockout button 38 is actuated to move interlock 70 out of engagement with knife driver 52. Interlock 70 is shown as fixedly secured to knife lockout button 38 about a centrally positioned pivot 72 so that user depressing of knife lockout button 38 causes a detent 74 of interlock 70 to pivot out of engagement with a corresponding notch 76 in knife driver 52. As is known, interlock 70 may be biased into the locked position and moveable to release knife driver 52 in response to a predetermined amount of user force applied to knife lockout button 38. In this manner, a user can select whether to use vessel sealer 10 without or without operation of knife 54 simply by applying pressure to knife lockout button 38.

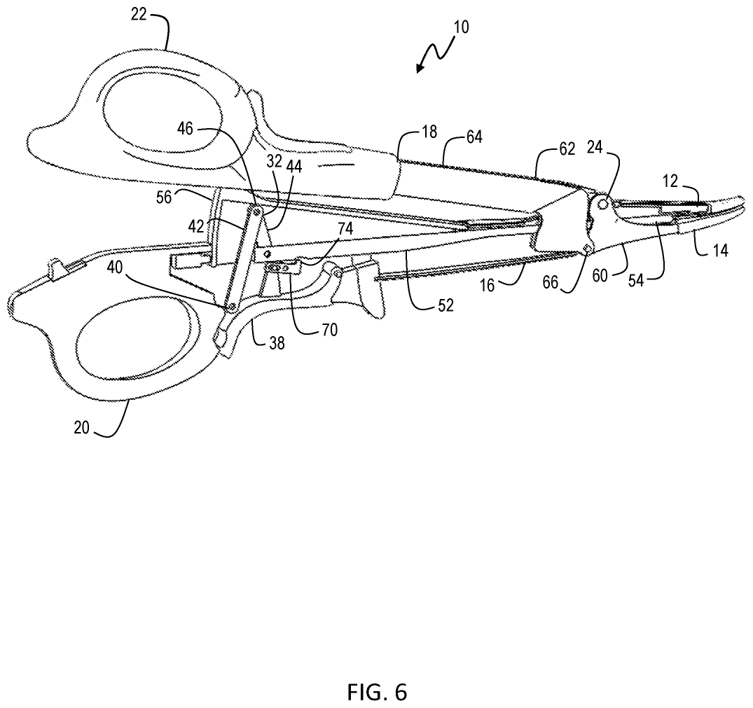

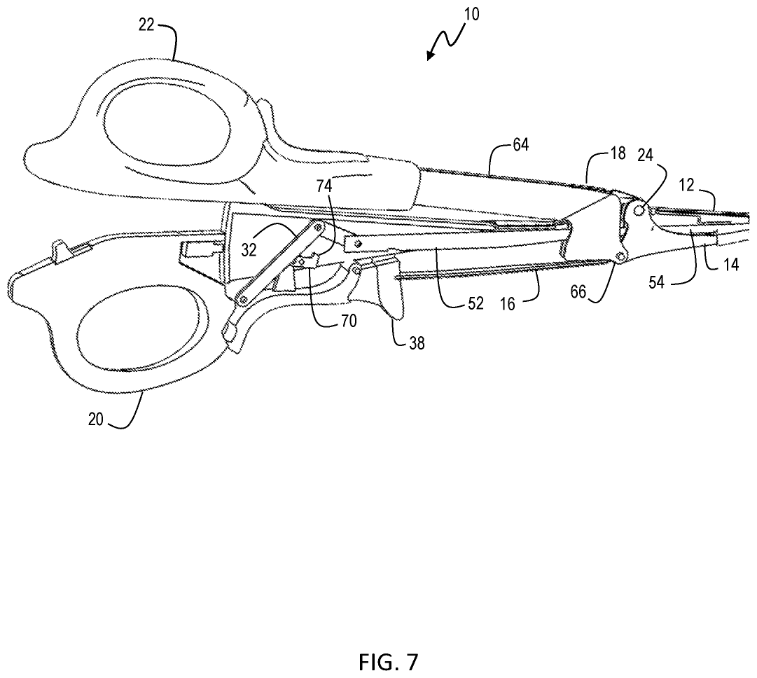

As seen in FIG. 6, when knife lockout button 38 is not actuated, scissoring of handles 20 and 22 will stop when handle 22 impacts cover 56 as linkage 32 will be prevented from movement from a retracted position to an extended position. As seen in FIG. 7, when lockout button 38 is actuated, handles 20 and 22 can be scissored beyond contact with cover 56 to drive linkage 32 and thus advance knife 54 between the closed jaws 12 and 14. It should be recognized that interlock 70 can be implemented using various structure such as the pivoting element show in FIGS. 5 through 7, and may be biased into the locked position. Alternatively, interlock 70 could be a spring loaded post that is withdrawn by movement of knife lockout button 38 and the biasing could be reversed so that knife driver 52 is allowed to advance unless knife lockout button 38 is actuated.

What is claimed is:

1. An electrosurgical device, comprising:
a first shaft having a first jaw positioned at a first end;
a second shaft pivotally coupled to the first shaft and having a second jaw associated with a second end so that the first shaft and second shaft are pivotal relative to each other to move the first jaw and the second jaw between an open position and a closed position;
a knife actuating assembly coupled to the first shaft and positioned to move a knife between a retracted position and an extended position between the first jaw and the second jaw in response to contact by the second shaft.

2. The electrosurgical device of claim 1, wherein the knife actuating assembly includes a linkage extending along the first shaft and secured to a knife pin.

3. The electrosurgical device of claim 2, wherein the knife pin is positioned in a slot extending longitudinally along the first shaft.

4. The electrosurgical device of claim 3, wherein the linkage includes a first leg pivotally coupled to the first shaft by a first pivot and a second leg coupled to the first leg by a second pivot.

5. The electrosurgical device of claim 4, wherein the second leg of the linkage is coupled to the knife pin.

6. The electrosurgical device of claim 5, wherein the second leg of the linkage is coupled to the knife.

7. The electrosurgical device of claim 1, wherein the second shaft includes a first section pivotally coupled to a second section.

8. The electrosurgical device of claim 7, wherein a spring interconnects the first section and the second section to prevent pivotal movement of the first section relative to the second section until the first jaw and the second jaw are in the closed position.

9. The electrosurgical device of claim 1, further comprising a knife lockout interconnected to the knife actuating assembly and moveable to selectively prevent or allow the knife actuating assembly to move the knife between the retracted position and the extended position.

10. The electrosurgical device of claim 9, wherein the knife lockout comprises an interlock secured to a knife lockout button that is accessible by a user.

11. The electrosurgical device of claim 10, wherein the knife lockout is pivotally mounted to the first shaft for movement between a first position where the interlock is in engagement with the knife actuating assembly and a second position where the interlock is out of engagement with the knife actuating assembly.

12. The electrosurgical device of claim 11, wherein the interlock includes a detect and the knife actuating assembly includes a notch into which the detent may fit.

13. The electrosurgical device of claim 12, wherein the detent is positioned in the notch when the first jaw and the second jaw are in the open position.
